(19) United States
(12) Patent Application Publication
(10) Pub. No.: US 2023/0255716 A1
(43) Pub. Date: Aug. 17, 2023

(54) PROBES TO DETECT TISSUE RESISTANCE DURING INSERTION

(71) Applicant: PROCEPT BioRobotics Corporation, Redwood City, CA (US)

(72) Inventors: Surag MANTRI, East Palo Alto, CA (US); Kevin Patrick STAID, Lowell, MA (US); Peter BENTLEY, Reno, NV (US)

(73) Assignee: PROCEPT BioRobotics Corporation, Redwood City, CA (US)

(21) Appl. No.: 17/816,321

(22) Filed: Jul. 29, 2022

Related U.S. Application Data
(60) Provisional application No. 63/268,176, filed on Feb. 17, 2022.

(51) Int. Cl. A61B 90/00

(52) U.S. Cl. CPC A61B 90/06; A61B 2090/065

(57) ABSTRACT

A probe for insertion into a patient comprises an elongate body. The elongate body comprises a distal portion and a proximal portion, in which the distal portion is shaped for insertion into the patient, and the proximal portion is coupled to the distal portion to advance the distal portion with advancement of the proximal portion. One or more sensors are supported with the elongate probe body and coupled to the distal portion of the probe to detect tissue resistance of the distal portion of the probe during insertion of the probe. An output is operatively coupled to the one or more sensors to provide feedback to a user in response to the tissue resistance. This output allows the user to respond and decrease pressure to the tissue. In some embodiments, a sheath comprising the one or more sensors is configured for placement over the probe prior to insertion.

### RELATED APPLICATIONS

The present application claims priority to U.S. Provisional Pat. App. 63/268,176, entitled “PROBES TO DETECT TISSUE RESISTANCE DURING INSERTION”, filed on Feb. 17, 2022, the entire disclosure of which is incorporated herein by reference.

The subject matter of the present application is related to PCT/US2015/048695, filed on Sep. 4, 2015, entitled “PHYSICIAN CONTROLLED TISSUE RESECTION INTEGRATED WITH TREATMENT MAPPING OF TARGET ORGAN IMAGES”, published as WO 2016/037137, on Mar. 10, 2016; PCT/US2020/021756, filed on Mar. 9, 2020, entitled “ROBOTIC ARMS AND METHODS FOR TISSUE RESECTION AND IMAGING”, published as WO/2020/181290 on Sep. 10, 2020; PCT/US2020/021708, filed on Mar. 9, 2020, entitled “STIFF SHEATH FOR IMAGING PROBE”, published as WO/2020/181280 on Sep. 10, 2020; PCT/US2020/058884, filed on Nov. 4, 2020, entitled “SURGICAL PROBES FOR TISSUE RESECTION WITH ROBOTIC ARMS”, published as WO/2021/096741 on May 20, 2021; and PCT/US2021/038175, filed on Jun. 21, 2021, entitled “SYSTEMS AND METHODS FOR DEFINING AND MODIFYING RANGE OF MOTION OF PROBE USED IN PATIENT TREATMENT”, published as WO/2021/262565 on Dec. 30, 2021; the entire disclosures of which are incorporated herein by reference.

### BACKGROUND

Surgical probes are used in many types of surgical procedures. At least some surgical procedures rely on the insertion of a probe into a naturally occurring lumen or body cavity. Work in relation to the present disclosure suggests that there can be a risk of unintended perforation with at least some surgical procedures, in which a natural tissue wall is unintentionally perforated with a probe, such as surgical probe or an imaging probe. For example, transrectal ultrasound (TRUS) can be used to image tissue during surgery such as prostate surgery. While extremely helpful for imaging tissue during surgery, there is a potential risk that a probe such as a transrectal ultrasound will perforate the wall of the rectum or colon in at least some instances. At least some natural lumens may comprise a pockets along the lumen, which may undesirably limit movement of the probe along the lumen, and work in relation to the present disclosure suggests that it may be helpful to detect tissue resistance prior to perforating the tissue.

In light of the above, there is a need for improved systems and methods for detecting tissue resistance to insertion of a probe or sheath that would ameliorate at least some of the aforementioned limitations of the prior approaches.

### SUMMARY

Embodiments of the present disclosure are directed to the detection of tissue resistance with one or more of a probe, a cover or a sheath configured for insertion into tissue, in order to decrease possible damage during insertion, such as perforation of a luminal wall. In some embodiments, the one or more of the probe, the cover or the sheath is configured for insertion into a patient and comprises an elongate body. The elongate body comprises a distal portion and a proximal portion, in which the distal portion is shaped for insertion into the patient, and the proximal portion is coupled to the distal portion to advance the distal portion with advancement of the proximal portion. One or more sensors are supported with the elongate body and coupled to the distal portion of the elongate body to detect tissue resistance of the distal portion related to advancement of the proximal portion of the body. An output is operatively coupled to the one or more sensors to provide feedback such as an alert to a user in response to the tissue resistance. In some embodiments, this output allows the user to respond and decrease pressure to the tissue from the distal end of the one or more of the probe, the cover or the sheath.

In some embodiments, a cover is configured to detect tissue resistance of a probe inserted into a patient while the probe is inserted into the patient. The probe can be any suitable probe such as a surgical probe or an imaging probe such as an ultrasound probe or an endoscope. The cover placed over the probe can allow the user to insert the probe and to detect tissue resistance while the probe is inserted. In some embodiments, the cover comprising an elongate sheath comprising a distal portion and a proximal portion configured to be placed on the probe, and one or more sensors supported with the sheath to detect a tissue resistance of the probe related to advancement of the probe into the patient. In some embodiments, the cover comprises a stiff sheath configured for advancement into the patient without the probe, such that the probe such as a treatment probe or imaging probe can be placed in the stiff sheath subsequent to placement of the stiff sheath in the patient.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a front view of a system for performing tissue resection in a patient, in accordance with some embodiments;

FIG. 2 schematically illustrates a system for performing tissue resection in a patient, in accordance with some embodiments;

FIGS. 3A and 3B show perspective views of a common base or mount for supporting one or more robotic arms, in accordance with some embodiments;

FIGS. 4A and 4B illustrate a perspective and side view, respectively, of a system for performing tissue resection in a patient that comprises a mobile base, in accordance with some embodiments;

FIG. 5A shows a probe inserted into tissue without significant tissue resistance, in accordance with some embodiments;

FIG. 5B shows a probe inserted into tissue with resistance, in accordance with some embodiments;

FIG. 6A shows a probe configured to limit force applied to tissue with a spring coupling a distal portion of the probe to a proximal portion of the probe, in accordance with some embodiments;

FIG. 6B shows a spring configured to provide a substantially constant force with a probe as in FIG. 6A, in accordance with some embodiments;

FIG. 7 shows a probe comprising one or more sensors configured to measure displacement between a proximal portion of the probe and a distal portion of the probe, in accordance with some embodiments;

FIG. 8A shows a probe comprising one or more sensors coupled to a waist of the probe to increase sensitivity to tissue resistance, in accordance with some embodiments;

FIG. 8B shows a probe comprising one or more sensors located between a first transducer array and a second transducer array, in accordance with some embodiments;

FIG. 9 shows a probe comprising one or more optical sensors, in accordance with some embodiments;

FIG. 10A shows a probe comprising one or more pressure sensors coupled to a pressure transducer, in accordance with some embodiments;

FIG. 10B shows an end view of an annular sensor of the probe of FIG. 10A, in accordance with some embodiments.

FIG. 11A shows a probe comprising an array of sensors to map tissue pressure on the probe, in accordance with some embodiments;

FIG. 11B shows a sensor on a tip of a probe, in accordance with some embodiments;

FIG. 12A shows a pressure sensing cover configured for placement on a probe, in accordance with some embodiments;

FIG. 12B shows a probe configured to receive a cover as in FIG. 12A, in which the probe comprises a connector to couple the cover to the probe, in accordance with some embodiments;

FIG. 12C shows a cover as in FIG. 12A placed over a probe as in FIG. 12B, in accordance with some embodiments;

FIG. 13 shows a probe configured to provide mechanical tactile feedback in response to tissue resistance, in accordance with some embodiments;

FIG. 14 shows a probe comprising a switch configured to transition between open and closed configurations in response to tissue resistance, in accordance with some embodiments; and

FIG. 15 shows a probe configured to measure tissue impedance and detect changes in tissue impedance in response to tissue resistance, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed systems and methods are well suited for use with many probes and diagnostic and surgical procedures. Although reference is made to a probe comprising a transrectal ultrasound probe inserted transrectally into a colon, the present disclosure is well suited for use with many types of probe inserted into many types of tissues, cavities and lumens, such as vascular lumens, nasal lumens and cavities, urethral lumens, gastric lumens, airways, esophageal lumens, trans esophageal, intestinal lumens, anal lumens, vaginal lumens, trans abdominal, abdominal cavities, kidney surgery, ureter surgery, kidney stones, prostate surgery, tumor surgery, cancer surgery, brain surgery, heart surgery, eye surgery, liver surgery, gall bladder surgery, bladder surgery, spinal surgery, orthopedic surgery arthroscopic surgery, liposuction, colonoscopy, intubation, minimally invasive incisions, minimally invasive surgery, and others.

The presently disclosed systems and methods are well suited for combination with prior probes such as imaging probes and treatment probes. Examples of such probes include laser treatment probes, water jet probes, RF treatment probes, microwave treatment probes, radiation therapy probes, ultrasound treatment probes, high intensity ultrasound treatment probes, phaco emulsification probes, imaging probes, endoscopic probes, resectoscope probes, ultrasound imaging probes, A-scan ultrasound probes, B-scan ultrasound probes, Doppler ultrasound probes, transrectal ultrasound probes, sagittal plane ultrasound imaging probes, transverse plane ultrasound imaging probes, and transverse and sagittal plane ultrasound imaging probes, for example.

The presently disclosed systems and methods are well suited for use with tissue comprising folds in a tissue wall, such as intestinal tissue, vaginal tissue, nasal tissue and conjunctival tissue. The presently disclosed systems and methods are well suited for protecting tissues from tearing, abrasion and perforation from forces related to insertion of the probe, which in some instances can be related to the probe engaging a fold of tissue.

In some embodiments, the probe is configured to limit forces to the distal tip, which can substantially decrease the probability of tissue damage. Alternatively or in combination, the probe can be configured to provide an alert, and may be coupled to another apparatus. The alert may comprise a graded alert, such as continuously variable scale, a variable tone, color bar graph, radial dial. In some embodiments, the alert comprises an audible tone that increases or decreases in frequency in response to pressure to the tissue. Alternatively or in combination, the alert may comprise a tactile vibration in response to the tissue resistance exceeding a threshold amount.

The sensor output such as an alert can be helpful for a user such as a physician during insertion of the probe. In some embodiments, the user is able to view data from the sensors while inserting the probe, and can determine how to proceed in response to the output data such as an alert.

In some embodiments, the alert comprises a binary configuration, e.g. on or off, configured to alert the user if a force threshold is met or exceeded. In some embodiments, the alert comprises one or more of an audible alarm, a visual cue, a flashing screen, a color change, e.g. from green to red, or a tactile vibration.

In some embodiments, the probe comprises one or more sensors such as a motion sensor to measure probe movement relative to the tissue surface to determine if probe is sliding on tissue surface, for example a Doppler ultrasound sensor. The one or more sensors on the probe may comprise any suitable sensor such as one or more of a fluidic sensor, a membrane, a membrane coupled to a fluid, a fluidic channel, a fluidic channel coupled to an electrical detector, a fluidic channel coupled to an electrical switch, a fluidic channel coupled to a pressure sensor, a fluidic sensor coupled to a proximal balloon, force sensor, a pressure sensor, a piezo electric sensor, a hall effect sensor, a capacitive sensor, an optical sensor, a strain gauge, a displacement transducer, a linear voltage displacement transducer (LVDT), an optical sensor, a light emitting diode, a diode laser, a photo diode, a photo transistor, a quadrant photo detector, a motion sensor, an accelerometer, an inertial measurement unit (IMU), Doppler ultrasound or tissue impedance sensor.

In some embodiments, the one or more sensors comprises a force transducer on a shaft of the probe to measure forces along one or more directions, such as along two or more dimensions or three or more dimension. The one or more sensors can be located at any suitable location of the probe, such as adjacent to a proximal handle of or a distal tip of the probe or to measure one or more of strain, pressure or forces along 3 axis, such as x, y and z axes of the probe. The one or more axes may correspond to axial bending relative to an elongate axis of the probe, axial displacement of the probe along the elongate axis of the probe, e.g. advancement and retraction of the probe.

In some embodiments, the one or more sensors is located on a distal portion of the probe such as a tip of the probe.

In some embodiments, the sensor is configured to detect displacement between a distal portion of the probe and a proximal portion of the probe.

In some embodiments, the sensor comprises an impedance sensor configured to measure a change in impedance of the tissue associated with pressure to the tissue.

In some embodiments, the sensor comprises channels with edges and a conductor at a bottom of the channel so that when tissue touches the conductor the tissue closes a circuit to trigger the alert.

In some embodiments, the one or more sensors comprises a balloon located on an end of the probe to detect a change of pressure within the balloon in response to tissue resistance. The balloon may comprise a compliant membrane or a non-compliant membrane over a chamber fluidically connected to a transducer comprising a pressure sensor.

In some embodiments, the one or more sensors is configured to be calibrated to provide a calibrated output signal in response to a calibrated input to the sensor such as a force or pressure to the sensor. In some embodiments, the one or more sensors comprise a calibrated spring coupled to an electrical switch configured to close (or open) a circuit in response to a calibrated pressure.

In some embodiments, the probe comprises a proximal portion and a distal tip portion configured to move independently of each other in response to tissue resistance. In some embodiments the distal portion and proximal portion are coupled to each other with a break away element configured to allow the distal portion to move independently of the proximal portion in response to the force to the distal portion exceeding a threshold amount. The break-away element can be configured with one or more of a linear configuration, an axial slide configuration, or an angular configuration. The break-away element can be configured to provide a user perceptible vibration such as an audible click or tactile haptic vibration.

In some embodiments, the one or more sensors on the probe can be used to place an imaging probe such as an ultrasound probe into engagement with the tissue to provide improved imaging. In some embodiments, an ultrasound probe is placed against the tissue to provide acoustic coupling with the tissue in response to the pressure of the probe engaging the tissue. Work in relation to the present disclosure suggests that appropriate amounts of pressure against the tissue can lead to improved ultrasound imaging, and the presently disclosed systems, probes and methods allow a user to place an ultrasound probe in engagement with the tissue with an appropriate amount of pressure in order to improve engagement with the tissue. In some embodiments, pressure mapping of the engagement of the ultrasound probe with the tissue can allow the user to place the probe with more uniform tissue pressure across the probe so as to provide improved acoustic coupling and acoustic engagement of the probe with the tissue.

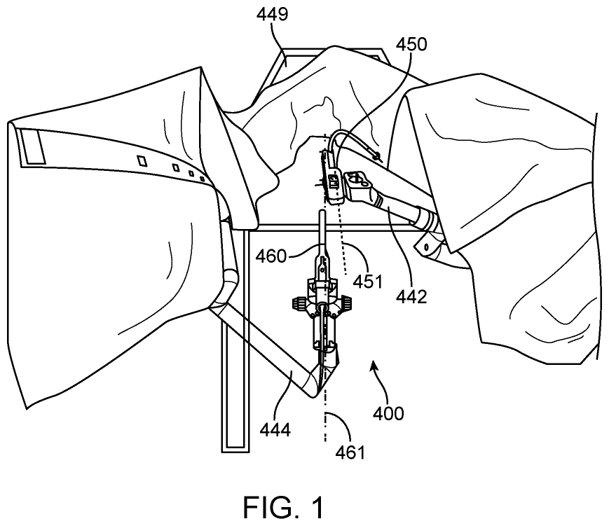

FIG. 1 shows an exemplary embodiment of a system 400 for performing tissue resection in a patient. The system 400 may comprise a treatment probe 450 and an imaging probe 460. The treatment probe 450 may be coupled to a first arm 442, and the imaging probe 460 coupled to a second am 444. One or both of the first arm 442 and the second arm 444 may comprise robotic arms, also referred to herein as instrument device manipulators, whose movements may be controlled by one or more computing devices operably coupled with the arms. The treatment probe 450 may comprise a device for performing any suitable diagnostic or treatment procedure, and may include resecting, collecting, ablating, cauterizing, or a combination of these or other treatments to tissue from a target site within a patient. The treatment probe 450 may be configured to deliver energy from the treatment probe 450 to the target tissue sufficient for removing the target tissue. For example, the treatment probe 450 may comprise an electrosurgical ablation device, a laser ablation device, a transurethral needle ablation device, a water jet ablation device, or any combination thereof. The imaging probe 460 may be configured to deliver energy from the imaging probe 460 to the target tissue sufficient for imaging the target tissue. The imaging probe 460 may comprise an ultrasound probe, a magnetic resonance probe, an endoscope, or a fluoroscopy probe, for example. The first arm 442 and the second arm 444 may be configured to be independently adjustable, adjustable according to a fixed relationship, adjustable according to a user selected relationship, independently lockable, or simultaneously lockable, or any combination thereof. The first arm 442 and the second arm 444 may have multiple degrees of freedom, for example six degrees of freedom, to manipulate the treatment probe 450 and the imaging probe 460, respectively. The treatment system 400 may be used to perform tissue resection in an organ of a patient, such a prostate of a patient. The patient may be positioned on a patient support 449 such as a bed, a table, a chair, or a platform. The treatment probe 450 may be inserted into the target site of the patient along an axis of entry that coincides with the elongate axis 451 of the treatment probe. For example, the treatment probe 450 may be configured for insertion into the urethra of the patient, so as to position an energy delivery region of the treatment probe within the prostate of the patient. The imaging probe 460 may be inserted into the patient at the target site or at a site adjacent the target site of the patient, along an axis of entry that coincides with the elongate axis 461 of the imaging probe. For example, the imaging probe 460 may comprise a transrectal ultrasound (TRUS) probe, configured for insertion into the rectum of the patient to view the patient's prostate and the surrounding tissues. As shown in FIG. 1, the first arm 442 and the second arm 444 may be covered in sterile drapes to provide a sterile operating environment, keep the robotic arms clean, and reduce risks of damaging the robotic arms.

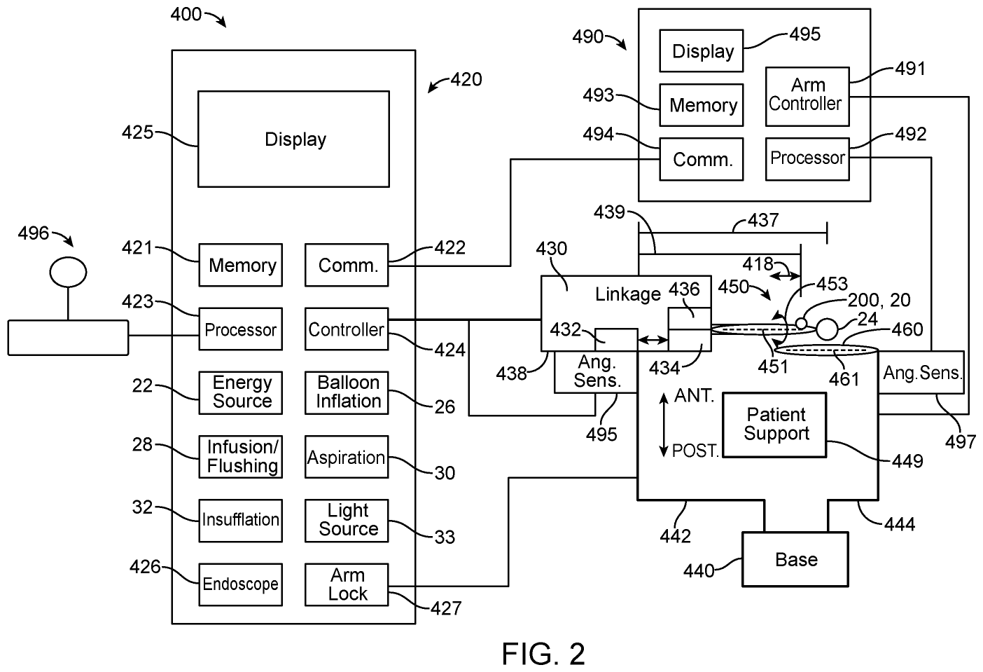

FIG. 2 schematically illustrates an exemplary embodiment of the system 400 for performing tissue resection in a patient. The system 400 comprises a treatment probe 450 and may optionally comprise an imaging probe 460. The treatment probe 450 is coupled to a console 420 and a linkage 430. The linkage 430 may comprise one or more components of the robotic arm 442. The imaging probe 460 is coupled to an imaging console 490. The imaging probe may be coupled to the second robotic arm 444, for example. The patient treatment probe 450 and the imaging probe 460 can be coupled to a common base 440. The patient is supported with the patient support 449. The treatment probe 450 is coupled to the base 440 with a first arm 442. The imaging probe 460 is coupled to the base 440 with a second arm 444. One or both of the first arm 442 and the second arm 444 may comprise robotic arms whose movements may be controlled by one or more computing devices operably coupled with the arms, as described in further detail herein.

Although reference is made to a common base, the robotic arms can be coupled to a bed rail, a console, or any suitable supporting structure to support the base of the robotic arm.

In some embodiments, system 400 comprises a user input device 496 coupled to processor 423 for a user to manipulate the surgical instrument on the robotic arm. A user input device 496 can be located in any suitable place, for example, on a console, on a robotic arm, on a mobile base, and there may be one, two, three, four, or more user input devices used in conjunction with the system 400 to either provide redundant avenues of input, unique input commands, or a combination. In some embodiments, the user input device comprises a controller to move the end of the treatment probe or the imaging probe with movements in response to mechanical movements of the user input device. The end of the probe can be shown on the display 425 and the user can manipulate the end of the probe. For example, the user input device may comprise a 6 degree of freedom input controller in which the user is able to move the input device with 6 degrees of freedom, and the distal end of the probe moves in response to movements of the controller. In some embodiments, the 6 degrees of freedom comprise three translational degrees of freedom and three rotational degrees of freedom. The processor can be configured with instructions for the probe control to switch between automated image guidance treatment with the energy source and treatment with the energy source with user movement of the user input device, for example.

The patient is placed on the patient support 449, such that the treatment probe 450 and ultrasound probe 460 can be inserted into the patient. The patient can be placed in one or more of many positions such as prone, supine, upright, or inclined, for example. In some embodiments, the patient is placed in a lithotomy position, and stirrups may be used, for example. In some embodiments, the treatment probe 450 is inserted into the patient in a first direction on a first side of the patient, and the imaging probe is inserted into the patient in a second direction on a second side of the patient. For example, the treatment probe can be inserted from an anterior side of the patient into a urethra of the patient, and the imaging probe can be inserted trans-rectally from a posterior side of the patient into the intestine of the patient. The treatment probe and imaging probe can be placed in the patient with one or more of urethral tissue, urethral wall tissue, prostate tissue, intestinal tissue, or intestinal wall tissue extending therebetween.

The treatment probe 450 and the imaging probe 460 can be inserted into the patient in one or more of many ways. During insertion, each of the first and second arms may comprise a substantially unlocked configuration such the treatment or imaging probe can be desirably rotated and translated in order to insert the probe into the patient. When the probe has been inserted to a desired location, the arm can be locked. In the locked configuration, the probes can be oriented in relation to each other in one or more of many ways, such as parallel, skew, horizontal, oblique, or non-parallel, for example. It can be helpful to determine the orientation of the probes with angle sensors as described herein, in order to map the image date of the imaging probe to treatment probe coordinate references. Having the tissue image data mapped to treatment probe coordinate reference space can allow accurate targeting and treatment of tissue identified for treatment by an operator such as the physician.

In some embodiments, the treatment probe 450 is coupled to the imaging probe 460 in order to align the treatment with probe 450 based on images from imaging probe 460. The coupling can be achieved with the common base 440 as shown. Alternatively or in combination, the treatment probe and/or the imaging probe may comprise magnets to hold the probes in alignment through tissue of the patient. In some embodiments, the first arm 442 is a movable and lockable arm such that the treatment probe 450 can be positioned in a desired location in a patient. When the probe 450 has been positioned in the desired location of the patient, the first arm 442 can be locked with an arm lock 427. The imaging probe can be coupled to base 440 with the second arm 444, which can be used to adjust the alignment of the imaging probe when the treatment probe is locked in position. The second arm 444 may comprise a lockable and movable arm under control of the imaging system or of the console and of the user interface, for example. The movable arm 444 may be micro-actuatable so that the imaging probe 460 can be adjusted with small movements, for example a millimeter or so in relation to the treatment probe 450.

In some embodiments, the treatment probe 450 and the imaging probe 460 are coupled to angle sensors so that the treatment can be controlled based on the alignment of the imaging probe 460 and the treatment probe 450. A first angle sensor 495 may be coupled to the treatment probe 450 with a support 438. A second angle sensor 497 may be coupled to the imaging probe 460. The angle sensors may comprise one or more of many types of angle sensors. For example, the angle sensors may comprise goniometers, accelerometers and combinations thereof. In some embodiments, the first angle sensor 495 comprises a 3-dimensional accelerometer to determine an orientation of the treatment probe 450 in three dimensions. In some embodiments, the second angle sensor 497 comprises a 3-dimensional accelerometer to determine an orientation of the imaging probe 460 in three dimensions. Alternatively or in combination, the first angle sensor 495 may comprise a goniometer to determine an angle of treatment probe 450 along an elongate axis 451 of the treatment probe. The second angle sensor 497 may comprise a goniometer to determine an angle of the imaging probe 460 along an elongate axis 461 of the imaging probe 460. The first angle sensor 495 is coupled to a controller 424 of the treatment console 420. The second angle sensor 497 of the imaging probe is coupled to a processor 492 of the imaging console 490. Alternatively or in combination, the second angle sensor 497 may be coupled to the controller 424 of the treatment console 420.

The console 420 comprises a display 425 coupled to a processor system in components that are used to control treatment probe 450. The console 420 comprises a processor 423 having a memory 421. Communication circuitry 422 is coupled to processor 423 and controller 422. Communication circuitry 422 is coupled to the imaging console 490 via the communication circuitry 494 of the imaging console. Arm lock 427 of console 420 may be coupled to the first arm 442 to lock the first arm or to allow the first arm to be freely movable to insert probe 450 into the patient.

Optionally, the console 420 may comprise components of an endoscope 426 that is coupled to anchor 24 of the treatment probe 450. Endoscope 426 can comprise components of console 420 and an endoscope insertable with treatment probe 450 to treat the patient.

Optionally, the console 420 may comprise one or more of modules operably coupled with the treatment probe 450 to control an aspect of the treatment with the treatment probe. For example, the console 420 may comprise one or more of an energy source 22 to provide energy to the treatment probe, balloon inflation control 26 to affect inflation of a balloon used to anchor the treatment probe at a target treatment site, infusion/flushing control 28 to control infusion and flushing of the probe, aspiration control 30 to control aspiration by the probe, insufflation control 32 to control insufflation of the target treatment site (e.g., the prostate), or a light source 33 such as a source of infrared, visible light or ultraviolet light to provide optical energy to the treatment probe.

The processor, controller and control electronics and circuitry can include one or more of many suitable components, such as one or more processor, one or more field-programmable gate array (FPGA), and one or more memory storage devices. In some embodiments, the control electronics controls the control panel of the graphic user interface (hereinafter “GUI”) to provide for pre-procedure planning according to user specified treatment parameters as well as to provide user control over the surgery procedure.

The treatment probe 450 may comprise an anchor 24. The anchor 24 can anchor the distal end of the probe 450 while energy is delivered to energy delivery region 20 with the probe 450. The probe 450 may comprise a nozzle 200.

The treatment probe 450 may be coupled to the first arm 442 with a linkage 430. The linkage 430 may comprise components to move energy delivery region 20 to a desired target location of the patient, for example, based on images of the patient. The linkage 430 may comprise a first portion 432, a second portion 434 and a third portion 436. The first portion 432 may comprise a substantially fixed anchoring portion. The substantially fixed anchoring portion 432 may be fixed to support 438. Support 438 may comprise a reference frame of linkage 430. Support 438 may comprise a rigid chassis or frame or housing to rigidly and stiffly couple the first arm 442 to treatment probe 450. The first portion 432 can remain substantially fixed, while the second portion 434 and third portion 436 can move to direct energy from the probe 450 to the patient. The first portion 432 may be fixed to the substantially constant distance 437 to the anchor 24. The substantially fixed distance 437 between the anchor 24 and the fixed first portion 432 of the linkage allows the treatment to be accurately placed. The first portion 432 may comprise a linear actuator to accurately position the high-pressure nozzle 200 in the energy delivery region 20 at a desired axial position along an elongate axis 451 of treatment probe 450.

The elongate axis 451 of treatment probe 450 generally extends between a proximal portion of the probe 450 near linkage 430 to a distal end having anchor 24 attached thereto. The third portion 436 can control a rotation angle 453 around the elongate axis 451. During treatment of the patient, a distance 439 between the energy delivery region 20 and the first portion 432 of the linkage may vary with reference to anchor 24. The distance 439 may adjust in manner 418 in response to computer control to set a target location along the elongate axis 451 of the treatment probe referenced to anchor 24. The first portion of the linkage remains fixed, while the second portion 434 adjusts the position of the energy delivery region 20 along the axis 451. The third portion of the linkage 436 adjusts the angle 453 around the axis in response to controller 424 such that the distance along the axis at an angle of the treatment can be controlled very accurately with reference to anchor 24. The probe 450 may comprise a stiff member such as a spine extending between support 438 and anchor 24 such that the distance from linkage 430 to anchor 24 remains substantially constant during the treatment. The treatment probe 450 is coupled to treatment components as described herein to allow treatment with one or more forms of energy such as mechanical energy from a jet, electrical energy from electrodes or optical energy from a light source such as a laser source. The light source may comprise infrared, visible light or ultraviolet light. The energy delivery region 20 can be moved under control of linkage 430 such as to deliver an intended form of energy to a target tissue of the patient.

The imaging console 490 may comprise a memory 493, communication circuitry 494 and processor 492. The processor 492 in corresponding circuitry is coupled to the imaging probe 460. An arm controller 491 is coupled to arm 444 to precisely position imaging probe 460. The imaging console may further comprise a display 495-1.

In order to facilitate precise control of the treatment probe and/or the imaging probe during treatment of the patient, each of the treatment probe and the imaging probe may be coupled to a robotic, computer-controllable arm. For example, referring to system 400 shown in FIG. 2, one or both of the first arm 442 coupled to the treatment probe 450 and the second arm 444 coupled to the imaging probe 460 may comprise robotic, computer-controllable arms. The robotic arms may be operably coupled with one or more computing devices configured to control movement of the robotic arms. For example, the first robotic arm 442 may be operably coupled with the processor 423 of the console 420, or the second robotic arm 444 may be operably coupled with the processor 492 of the imaging console 490 and/or to the processor 423 of the console 420. The one or more computing devices, such as the processors 423 and 492, may comprise computer executable instructions for controlling movement of the one or more robotic arms. The first and second robotic arms may be substantially similar in construction and function, or they may be different to accommodate specific functional requirements for controlling movement of the treatment probe versus the imaging probe.

Either or both robotic arms may comprise 6 or 7 or more joints to allow the arm to move under computer control. Suitable robotic arms are commercially available from several manufacturers such as RoboDK Inc., Kinova Inc. and several other manufacturers.

The one or more computing devices operably coupled to the first and second robotic arms may be configured to automatically control the movement of the treatment probe and/or the imaging probe. For example, the robotic arms may be configured to automatically adjust the position and/or orientation of the treatment probe and/or imaging probe during treatment of the patient, according to one or more pre-programmed parameters. The robotic arms may be configured to automatically move the treatment probe and/or imaging probe along a pre-planned or programmed treatment or scanning profile, which may be stored on a memory of the one or more computing devices. Alternatively or additionally to automatic adjustment of the robotic arms, the one or more computing devices may be configured to control movement of the treatment probe and/or the imaging probe in response to user inputs, for example through a graphical user interface of the treatment apparatus. Alternatively or additionally to automatic adjustment of the robotic arms, the one or more computing devices may be configured to control movement of the treatment probe and/or the imaging probe in response to real-time positioning information, for example in response to anatomy recognized in one or more images captured by the imaging probe or other imaging source (from which allowable ranges of motion of the treatment probe and/or the imaging probe may be established) and/or position information of the treatment probe and/or imaging probe from one or more sensors coupled to the probes and/or robotic arms.

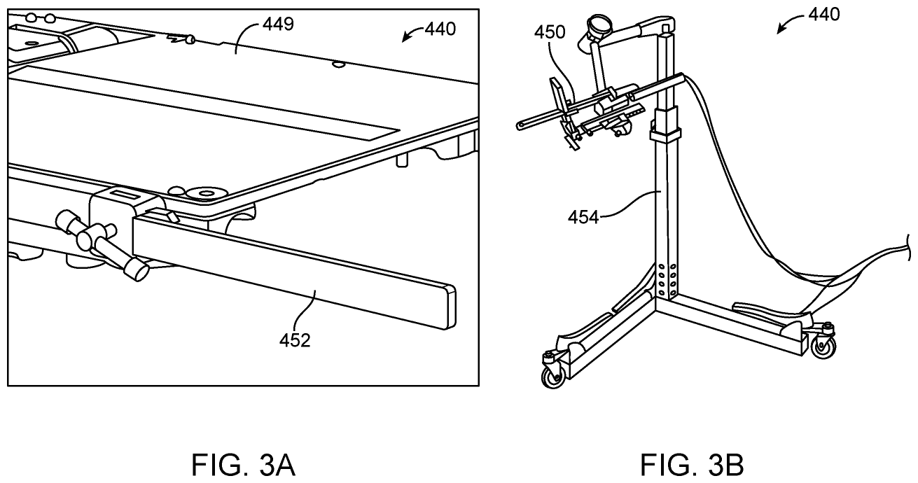

FIGS. 3A and 3B show exemplary embodiments of a common base or mount 440 for supporting one or more robotic arms of an image-guided treatment system as disclosed herein. FIG. 3A shows a patient support 449 comprising one or more rails 452. The patient support 449 may comprise a surgical table or a platform. One or more robotic arms associated with one or more of the treatment probe or the imaging probe may be mounted to the rails 452, such that the rails function as the common base 440. FIG. 3B shows a common base 440 comprising a floor stand 454 configured to couple to the first robotic arm connected to the treatment probe and/or the second robotic arm connected to the imaging probe. The floor-stand 454 may be positioned between the patient's legs during the treatment procedure.

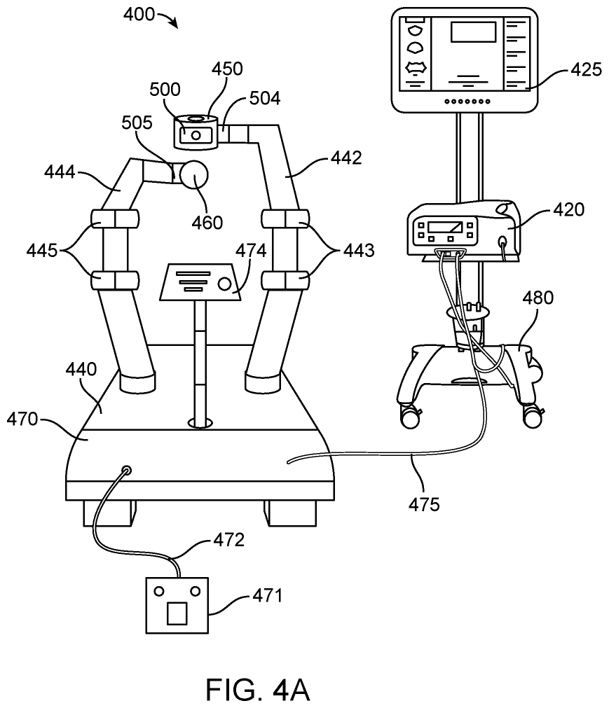

FIGS. 4A and 4B illustrate an exemplary embodiment of a treatment system 400 as described herein comprising a mobile base 470. FIG. 4A is a front view and FIG. 4B is a side view of the treatment system 400. The treatment system 400 comprises a treatment probe 450 coupled to a first robotic arm 442, and an imaging probe 460 coupled to a second robotic arm 444. The first robotic arm 442 and the second robotic arm 444 each comprises a proximal end and a distal end, the distal end coupled to the treatment probe 450 and the imaging probe 460, respectively, and the proximal end coupled to a common base 440 comprising a mobile base 470. The first robotic arm 442 may comprise a first arm coupling structure 504 to couple to the treatment probe 450, and the second robotic arm 444 may comprise a second arm coupling structure 505 to couple to the imaging probe 460. The treatment probe 450 may be coupled to the distal end of the first robotic arm 442 via an attachment device 500, which may comprise a linkage configured to affect movement of the treatment probe as described herein (e.g., rotation, translation, pitch, etc.). Coupling of the treatment probe 450 to the first robotic arm 442 may be fixed, releasable, or user adjustable. Similarly, coupling of the imaging probe 460 to the second robotic arm 444 may be fixed, releasable, or user adjustable.

The first robotic arm 442 may articulate at one or more first arm joints 443. The imaging arm 444 may articulate at one or more second arm joints 445. Each arm joint 443 or 445 may be operably coupled with a computer-controllable actuator, such as a stepper motor, to affect movement at the joint. Each arm joint 443 or 445 may comprise one of a variety of kinematic joints including but not limited to a prismatic, revolute, parallel cylindrical, cylindrical, spherical, planar, edge slider, cylindrical slider, point slider, spherical slider, or crossed cylindrical joint, or any combination thereof. Moreover, each arm joint 443 or 445 may comprise a linear, orthogonal, rotational, twisting, or revolving joint, or any combination thereof.

The system 400 may further comprise a console 420 as described herein, which may be supported by a mobile support 480 separate from the mobile base 470. The console 420 may be operably coupled with the mobile base 470 via a power and communication cable 475, to allow control of the treatment probe 450 coupled to the mobile base via the first robotic arm. The treatment console 420 comprises a processor and a memory having stored thereon computer-executable instructions for execution by the processor, to control various modules or functionalities of the treatment console, such as an energy source, infusion/flushing control, aspiration control, and other components as described herein with reference to FIG. 2. The treatment console 420 may further comprise a display 425 in communication with the processor. The display 425 may be configured to display, for example, one or more of: subject vital signs such as heart rate, respiratory rate, temperature, blood pressure, oxygen saturation, or any physiological parameter or any combination thereof; status of a procedure; one or more previously taken images or sequence of images of a treatment site from one or more views; one or more real-time images or sequence of images of the treatment site from one or more views acquired by the imaging probe 460; a set of treatment parameters including but not limited to a treatment mode such as cutting or coagulating, an intensity of treatment, time elapsed during treatment, time remaining during treatment, a depth of treatment, an area or volume of the treatment site that has been treated, an area of the treatment site that will be treated, an area or volume of the treatment site that will not be treated, location information of the treatment probe 450 or the imaging probe 460 or both; treatment adjustment controls such as means to adjust the depth of treatment, the intensity of treatment, the location and/or orientation of the treatment probe 450, the depth of imaging, or the location and/or orientation of the imaging probe 460, or any combination thereof; or system configuration parameters.

The mobile base 470 may further comprise one or more computing devices to control operation of the one or more robotic arms. For example, the mobile base may comprise processors and a memory having stored thereon computer executable instructions for execution by the one or more processors. The memory may have stored thereon instructions for operating the one or more robotic arms coupled to the mobile base. The processor may be operably coupled with the robotic arms via suitable electromechanical components to affect movement of the robotic arms. For example, each of the one or more joints of a robotic arm may comprise a step motor, and the processor may be operably coupled with the step motor at each joint to actuate the motor by a specified increment in a specified direction. Alternatively, the one or more robotic arms may be operably coupled with one or more processors of the console 420 or a separate imaging console (such as imaging console 490 shown in FIG. 2), wherein the one or more console processors may be configured to execute instructions for controlling movement of the one or more robotic arms, and may communicate the instructions to the robotic arms via communication circuitry (such as communication circuitry 422 of console 420 or communication circuitry 494 of console 490 shown in FIG. 2). The computer executable instructions for controlling movement of the robotic arms may be pre-programmed and stored on a memory, or may be provided by a user via one or more user inputs before or during treatment of the patient using the treatment system.

The one or more computing devices operably coupled with the first and/or second robotic arms may be configured to control movement of the arms so as to adjust the pitch, yaw, roll, and/or linear position of the treatment probe and/or imaging probe along the target site.

The mobile base 470 may comprise one or more user input devices to enable a user to control movement of the robotic arms under computer instructions. For example, as shown in FIGS. 4A and 4B, the mobile base may comprise a keyboard 474 and/or a footswitch 471, the footswitch operably coupled with the mobile base via a footswitch cable 472. The keyboard 474 and the footswitch 471, independently or in combination, may be configured to control operation of the first robotic arm 442 and/or the second robotic arm 444, for example via articulation of one or both robotic arms at one or more joints. The keyboard and the footswitch may be in communication with the one or more processors configured to control movement of the robotic arms. When a user inputs instructions into the keyboard and/or the footswitch, the user instructions can be received by the one or more processors, converted into electrical signals, and the electrical signals may be transmitted to the one or more computer-controllable actuators operably coupled with the one or more robotic arms. The keyboard and/or the footswitch may control movement of one or both arms towards or away from a treatment position, a position of interest, a predetermined location, or a user-specified location, or any combination thereof.

Optionally, the keyboard 474 and the footswitch 471, independently or in combination, may be configured to control operation of the treatment probe 450 and/or imaging probe 460. For example, the keyboard 474 and/or footswitch 471 may be configured to start, stop, pause, or resume treatment with the treatment probe. The keyboard 474 and/or footswitch 471 may be configured to begin imaging or freeze, save, or display on the display 425 an image or sequence of images previously or currently acquired by the imaging probe.

The mobile base 470 and the mobile support 480 of the console 420 may be independently positionable around a patient, supported by a patient support 449 such as a platform. For example, the mobile base 470, supporting the first and second robotic arms and the treatment and imaging probes, may be positioned between the patient's legs, while the mobile support 480 carrying the console 420 and the display 425 may be positioned to the side of the patient, such as near the torso of the patient. The mobile base 470 or the mobile support 480 may comprise one or more movable elements that enable the base or the support to move, such as a plurality of wheels. The mobile base 470 may be covered with sterile draping throughout the treatment procedure, in order to prevent contamination and fluid ingress.

The probe such as TRUS probe 460 or treatment probe 450 as shown in FIGS. 1 to 4B can be advanced into the patient in many ways in accordance with the present disclosure. In some embodiments, the probe is advanced manually. The manual advancement may be provided by the physician pushing on the probe. Alternatively or in combination, the probe is advanced manually by the physician manipulating a proximal control such as a knob coupled to a rack and pinion drive to advance and retract the probe. In some embodiments, the manual drive comprises one or more sensors such as encoders or linear displacement voltage transducers (LVDTs) to measure movement of the probe during insertion and retraction of the probe.

In some embodiments, the one more sensors is coupled to a processor to receive displacement data such as axial displacement of the probe, and this displacement data is combined with tissue data to detect tissue resistance.

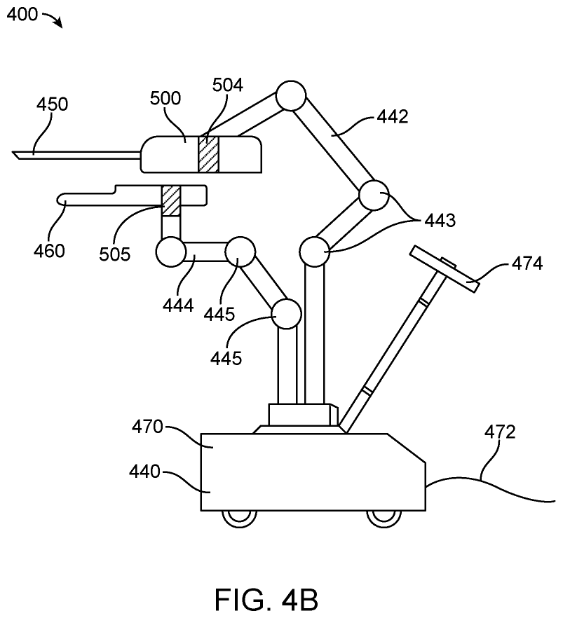

FIG. 5A shows a probe 510 inserted into tissue 502 without significant tissue resistance. The probe 510 comprises an elongate axis 511 and can be advanced in a direction along the elongate axis 511. The tissue 502 comprises a plurality of tissue structures 504. The tissue can be imaged with an ultrasound probe, for example. In some embodiments, the tissue 502 and tissue structures 504 are visible in a sagittal ultrasound image 501, for example when probe 510 comprises a TRUS probe as described herein. The movement of probe 520 may comprise any suitable movement such as advancement 522 or retraction 524. When probe 510 moves within a lumen 507 along a luminal wall 506 with relatively little resistance, the probe moves independently of the tissue and with relatively little resistance from the tissue. The velocity of the tissue structures in the image 501 correspond to the velocity of the probe and have a substantially uniform velocity gradient 503 that matches the insertion velocity of the probe, as shown with the arrows.

The probe 510 may comprise any suitable probe as described herein. In some embodiments, probe 510 comprises treatment probe 450. Alternatively probe 510 may comprise an imaging probe such as ultrasound probe 460. In some embodiments probe 510 comprises a component of a system 500. System 500 may comprise one or more components of system 400 as described herein.

FIG. 5B shows a probe inserted into tissue with a resistance 590 to advancement of the probe within the lumen 507 against tissue such as a luminal wall 506. The resistance can be related to several factors such as hydration of the lumen, shape of the lumen, the presence of gels within the lumen, friction, and other factors. In some embodiments, the tissue 502 comprises a fold 508, which can increase resistance 590 in response to advancement of the probe. The resistance 590 may comprise one or more of a force or a pressure related to advancement of the probe 510. The resistance of the tissue to the movement of the probe results in a non-uniform velocity gradient 503. In some embodiments, the tissue structures in the image located farther from the probe appear to move more quickly than the tissue structures closer to the probe, because the tissue structures located closer to the probe tend to move a least partially with the probe while the tissue structures located farther from the probe remain substantially fixed while the probe moves.

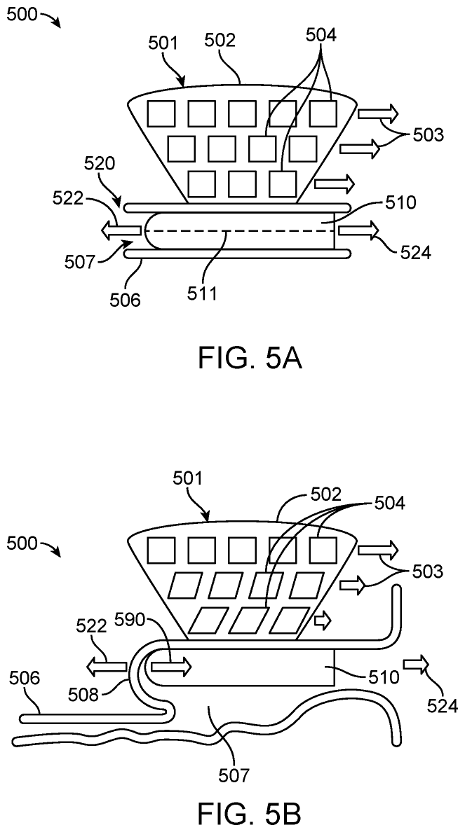

FIG. 6A shows a probe configured to limit force applied to tissue with a coupling 540 between a distal portion 514 of the probe 510 and a proximal portion 512 of the probe. In some embodiments the proximal portion 512 comprises a handle 519. In some embodiments, the coupling 540 is located between proximal portion 512 and distal portion 514. While the coupling 540 can be configured in any suitable way, in some embodiments coupling 540 comprises a resilient structure such as a spring 542. The spring may comprise a tensioned spring or compressed spring, for example.

In some embodiments, the proximal portion is configured to move relative to the distal portion when the resistance 590 exceeds an amount of force provided by spring 542. While the coupling 540 can be configured in many ways, in some embodiments, coupling 540 comprises a telescopic coupling which allows distal portion 514 to move in relation to proximal portion 512. The in some embodiments, the coupling 540 comprises stops 544, which limit movement of the proximal end away from the distal end, to allow the spring to provide the amount of force between the proximal end and the distal end. In some embodiments, the spring comprises sufficient tension or compression to provide the appropriate amount of force when the stops are engaged. When the amount of force to the tissue at the distal end of the probe exceeds the spring force, the proximal portion moves toward the distal portion to substantially limit the force to the tissue to the force provided by the spring. Because additional movement of the proximal end toward the distal end may increase pressure to the tissue related to compression of the spring, the spring may comprise a substantially constant force spring.

In some embodiments, the spring comprises a substantially fixed force spring. In some embodiments, the spring starts compressing in response to tissue resistance above a threshold amount. In some embodiments, the coupling 540 and spring 542 are configured to limit axial force to the distal tip related to advancement of the proximal portion. The spring can be configured in many ways and may comprise a constant force spring similar to a watch spring, as will be understood by one of ordinary skill in the art.

In some embodiments, the coupling is connected to a sensor such as a switch, which can be configured to provide an alert such as an alarm in response to the force to the spring above the threshold amount such that the relative distance between the proximal portion and the distal portion decreases.

Although FIG. 6A shows a coupling 540 in which the proximal portion 512 fits inside the distal portion 514, in some embodiments, this configuration is reversed and the distal portion 514 fits inside the proximal portion 512.

FIG. 6B shows a spring 542 configured to provide a substantially constant force with a probe as in FIG. 6A, in accordance with some embodiments.

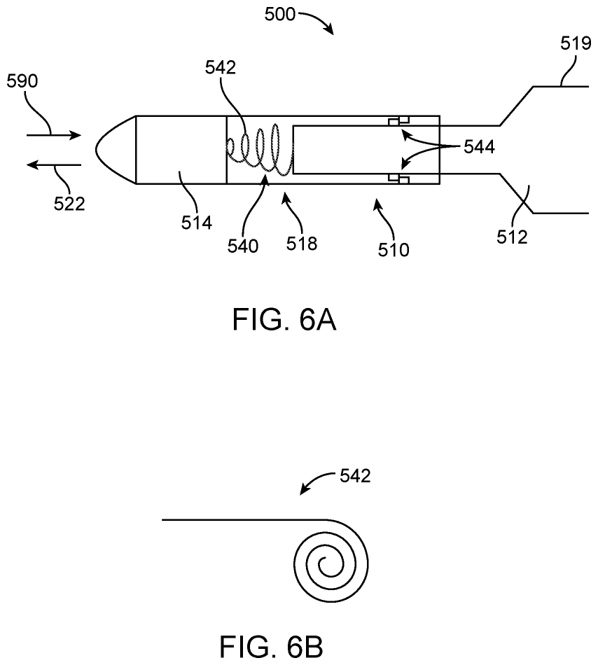

FIG. 7 shows probe 510 comprising one or more sensors 540 configured to measure displacement between a proximal portion of the probe 512 and a distal portion 514 of the probe 510. The probe 510 may comprise elements similar to probe 500 shown in FIGS. 6A and 6B, such as coupling 530.

The sensor 530 may comprise any suitable transducer, such as one or more of a fluidic sensor, a membrane, a membrane coupled to a fluid, a fluidic channel, a fluidic channel coupled to an electrical detector, a fluidic channel coupled to an electrical switch, a fluidic channel coupled to a pressure sensor, a fluidic sensor coupled to a proximal balloon, force sensor, a pressure sensor, a piezo electric sensor, a hall effect sensor, a capacitive sensor, an optical sensor, a strain gauge, a displacement transducer, a linear voltage displacement transducer (LVDT), an optical sensor, a light emitting diode, a diode laser, a photo diode, a photo transistor, a quadrant photo detector, a motion sensor, an accelerometer, an inertial measurement unit (IMU), Doppler ultrasound or tissue impedance sensor. In some embodiments, sensor 530 comprises an LVDT transducer configured to measure displacement between proximal portion 512 and distal portion 514. In some embodiments, displacement between the proximal portion and the distal portion is related to force delivered to the tissue with the tip on the distal portion 514.

The sensor 530 can be coupled to a processor to generate an alert to the user as described herein, such as when the force exceeds a threshold amount. In some embodiments, as the spring is compressed, the sensor 530 reports a greater intensity signal to the processor, in which the signal corresponds to the force on the spring and corresponding for to the tissue provided by the tip. In some embodiments, the transducer signal from the sensor triggers an alert comprising an alarm in response to the force applied to the tissue.

The sensor 530 and processor can be configured in any suitable way to generate an appropriate alert. In some embodiments, the sensor provides a substantially continuous readout value related to one or more of the force or pressure of the tip to the tissue, and the alert may comprise a corresponding substantially continuous readout value. In some embodiments, the processor is configured to generate an alert such as an alarm in response to the sensor read out value corresponding to a pressure or force above a threshold amount. In some embodiments, the sensor comprises a switch configured to transition between an open configuration and a closed configuration and to generate the alert in response a change in the configuration of the switch. For example, the closing of the switch circuit in response to pressure or force to the tip can trigger the alert such as an alarm.

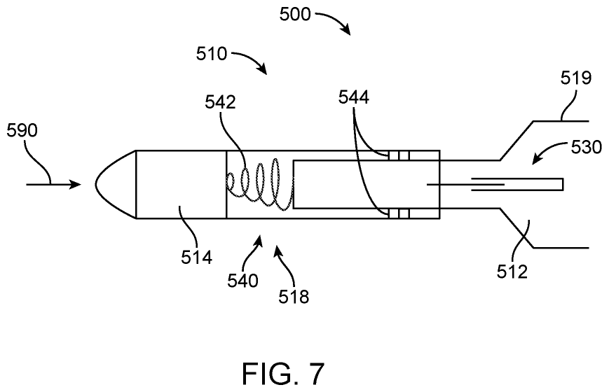

FIG. 8A shows a probe 510 comprising one or more sensors 530 coupled to a probe to measure one or more of axial or bending loads. In some embodiments, the one or more sensors comprises a first sensor to measure a bending load and a second sensor to measure an axial load. The one or more sensors 530 may comprise a plurality of sensors contained in a package to measure axial and bending loads, for example. In some embodiments, the one or more sensors comprises a first sensor arranged at a first angle in relation to an elongate axis of the probe and a second sensor arranged at a second angle in relation to an elongate axis of the probe to measure an axial load and a bending load.

In some embodiments probe 510 comprises waist 516 to increase sensitivity to tissue resistance measurements. Although reference is made to a waist 516 comprising a tapered portion of the probe, the one or more sensors 530 can be applied to any suitable location of the probe without a waist. In some embodiments, the waist 516 comprises a weakened portion of the probe at an intermediate portion 518 of the probe. The weakened portion can be configured in many ways and may comprise a weakened material, the waist, perforations, slits or other structures to allow movement of the proximal portion 512 relative to the distal portion 514 that can be measured with the one or more sensors 530.

In some embodiments, the one or more sensors comprises one or more strain gauges. In some embodiments, the one or more strain gauges comprises a plurality of strain gauges. While the plurality of strain gauges can be configured in many ways, in some embodiments, the plurality of strain gauges is located at a common axial position along the probe with different angular locations, so as to provide axial compression and deflection data. In some embodiments, the plurality of strain gauges, such as 4 strain gauges located circumferentially around the probe, such as at approximately 90 degrees to each other. Alternatively or in combination, the plurality of strain gauges may be located at a plurality of locations along the probe corresponding to a plurality of axial locations.

In some embodiments, probe 510 comprises an imaging probe such as an ultrasound imaging probe comprising an ultrasound array 550. The array 550 can be configured in many ways. In some embodiments, array 550 comprises a first array 552 for transverse imaging and a second array 554 for sagittal imaging. The one or more sensors 530 can arranged in any suitable way with respect to array 550. In some embodiments, the first array 552 and the second array 554 are located between the proximal portion 512 and the distal portion 514 and the one or more sensors 530 are located between the proximal portion 512 and the distal portion 514. In some embodiments, the one or more sensors 530 are located between the first array 552 and the second array 554. In some embodiments, the one or more sensors 530 are not located between the first array 552 and the second array 554. In some embodiments, the one or more sensors 530 are located distal to the first array 552 and the second array 554. In some embodiments, the one or more sensors 530 are located proximal to the first array 552 and the second array 554.

FIG. 8B shows a probe 510 comprising one or more sensors 530 located between a first transducer array 552 and a second transducer array 554. The one or more sensors may comprise a first sensor and a second sensor arranged to measure bending load and axial load, for example. In some embodiments, the first sensor and the second sensor are located within a package, for example.

The one or more sensors 530 on probe 510 can be arranged and configured in many ways. In some embodiments, the one or more sensors comprises a plurality of piezo electric transducers to measure load to the probe. In some embodiments, the plurality of transducers comprises a piezo electric force transducer grid placed over a surface of probe on one or more of proximal portion 512, distal portion 514 or intermediate portion 518 in order to measure one or more of axial loading or bend loading. In some embodiments, this configuration provides force data on each transducer element on where and how much load is applied to the probe 510.

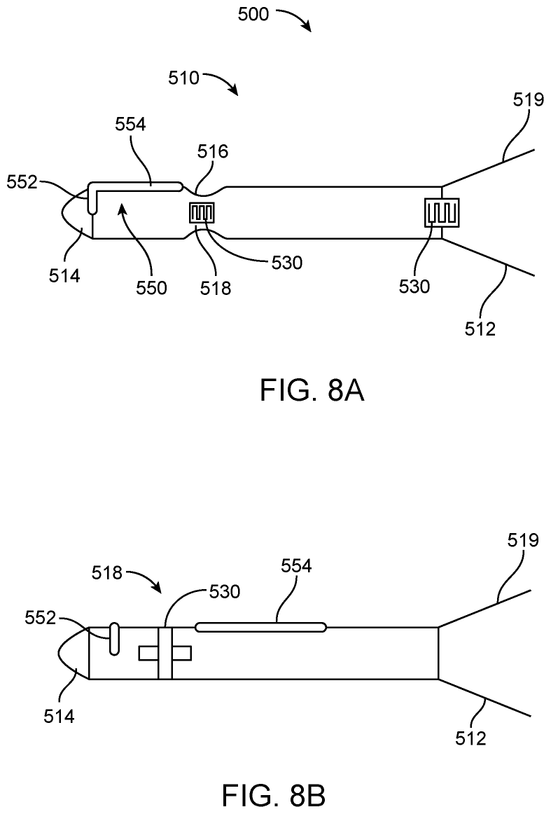

FIG. 9 shows a probe 510 in which the one or more sensors 530 comprises one or more optical sensors. The probe 510 may comprise one or more components as described herein, such as coupling 540 and intermediate portion 518 between proximal portion 512 and distal portion 514.

While the one or more optical sensors can be configured in many ways, in some embodiments the one or more optical sensors comprises one or more of a light emitting diode, a diode laser, a photo diode, a photo transistor, or a quadrant photo detector for example. The one or more optical sensors can be configured to measure displacement between proximal portion 512 and distal portion 514, for example with movement provided by coupling 540. The sensor 540 may comprise a reflective or scattering surface which changes an intensity of a light beam from a light source 910 such as a diode laser that is received by a detector 920 such as a quadrant photo detector.

In some embodiments, sensor 540 comprises a mirror connected to a back of the distal portion 514 comprising the tip, and the mirror is configured to moves axially and or tilt with the tip in response to forces to the tip. For example, by providing a suitable material within coupling 540 the free floating tip allows the tilt angle of the mirror to change in response to tilting of the tip from tissue forces to the tip. In some embodiments, a diode laser is directed toward the mirror, and a photo detector measures the change in angle of the laser which can be translated to tip movement laterally and axially.

Coupling 540 may comprise a suitable material to provide resistance to axial displacement, such as one or more of an elastic material, a flexible material or deformable material 543, in which the material 543 comprises any suitable material capable of providing resistance to forces when shaped appropriately, such as one or more of rubber, elastomer, spring metal, or plastic for example. Movement of the distal portion 514 relative to proximal portion 512 can be related to one or more of force or pressure to the tip of distal portion 512 as described herein.

In some embodiments, the tip of distal portion 514 is somewhat free to float within a shaft at coupling 540.

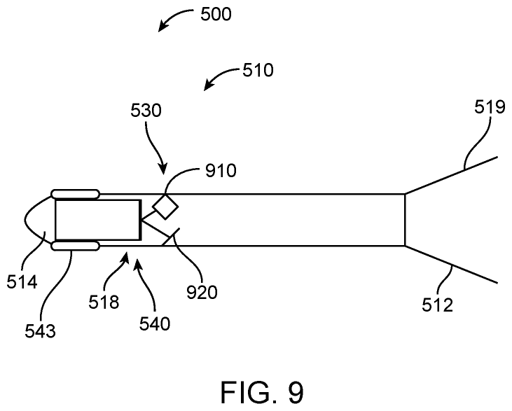

FIG. 10A shows a probe 510 in which the one or more sensors 530 comprises one or more pressure sensors 532 coupled to a pressure transducer 534. The one or more pressure sensors 532 can be coupled to the pressure transducer 534 with one or more lines 538 extending between the pressure sensors 532 and pressure transducer 534. The one or more pressure sensors 532 may comprise any suitable sensor as described herein, such as one or more fluidic pressure sensors. In some embodiments, the one or more pressure sensors 532 comprises one or more membranes fluidically coupled to the one or more pressure transducers 534 with one or more fluidic lines 538 extending therebetween, so as to transmit pressure from the one or more sensors to the one or more transducers with the one or more fluidic lines. In some embodiments, the one or more pressure sensors and the one or more fluidic lines comprise a substantially incompressible fluid, such as a liquid. In some embodiments, the one or more pressure sensors comprises an annular pressure sensor 531 located on distal portion 514.

In some embodiments, the one or more pressure sensors 532 comprises a plurality of pressure sensors 532 arrange to map tissue pressure on the tip of the distal portion 514. In some embodiments, the one or more lines 538 comprises a plurality of lines, e.g. a plurality of signal channels, extending between the plurality of pressure sensors 532 and the pressure transducer. In some embodiments, the plurality of pressure sensors and the plurality of lines are configured to independently measure the pressure of each of the pressure sensors. In some embodiments pressure transducer 534 comprises a multi-channel pressure transducer coupled to the plurality of lines and the plurality of pressure sensors in order to impudently measure the pressure of each of the plurality of sensors 532. In some embodiments, the plurality of sensors is located on the tip of the distal portion in order to map tissue pressure on the distal portion 514 of the probe 510. The map of pressure on the probe can be shown on a display, for example with a real time map showing colors in which hotter colors, e.g. red correspond to increased pressure, and cooler colors, e.g. blue correspond to lower amounts of pressure on the pressure map of the distal portion 514 of the probe 512.

The pressure sensor and lines can be configured in any suitable way. In some embodiments, channels are cut in a surface of the probe, and a flexible membrane covers channels and are filled with a fluid. The channels can be formed in any suitable way, and in some embodiments, the channels are etched with a process such as laser etching or lithography. The channels can be sized and shaped in any suitable manner to define the pressure sensors and the lines extending from the pressure sensors to the transducer. The membrane may comprise any suitable material such as a compliant or a non-compliant material similar to materials used with balloon catheter as will be known by one of ordinary skill in the art.

In some embodiments, the pressure in the chambers of the fluidic pressure sensor corresponds to the pressure of the membrane against the tissue and may comprise a pressure substantially equal to the pressure of the probe against tissue.

The pressure sensor can be coupled to a processor, and the pressure can be displayed continuously or used to generate an alert as described herein, for example.

Although reference is made to fluidic pressure sensors coupled to transducer with lines extending therebetween, in some embodiments a transducer can be located within each fluid chamber and coupled to a processor such as a signal processor with lines such as wires.

FIG. 10B shows an end view of probe 510, in which the one or more sensors 532 comprises one or more annular pressure sensors 531.

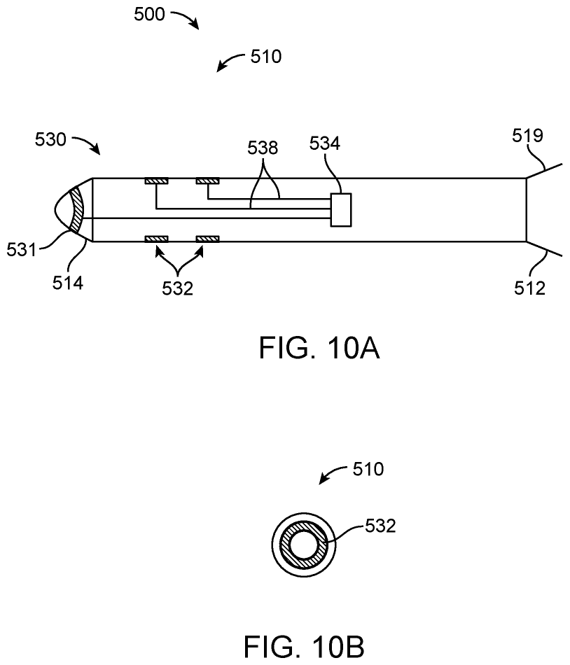

FIG. 11A shows a probe comprising a probe 510 in which the one or more sensors 530 comprises an array of sensors to map tissue pressure on the probe, in accordance with some embodiments. The one or more sensors may comprise a plurality of locations on the probe to determine pressure on the probe. The plurality sensors may comprise any suitable sensor. In some embodiments, the plurality of sensors is coupled to an electrical component such as a multiplexer or transducer 534 with a plurality of lines 538. In some embodiments, the plurality of sensors 530 comprises a plurality of capacitive sensors 536 coupled to a capacitance measurement circuit. In some embodiments, the capacitance of each sensor corresponds to the tissue pressure on the sensor, and the capacitance and pressure is measured independently in order to map the tissue pressure as described herein.

In some embodiments, the plurality of sensors comprises a plurality of piezo electric sensors. In some embodiments, the plurality of piezo electric sensors comprises a grid a piezo electric sensor places of at least the distal portion of the probe in order to map the tissue pressure on the probe.

FIG. 11B shows probe 510 in which the one or more sensors 530 comprises a sensor such as a pressure sensor, e.g. a fluidic sensor, on a tip of the distal portion 514 of the probe 510. The fluidic sensor 532 may comprise a channel and membrane coupled to a transducer 534 as described herein. The transducer can be coupled to a processor to provide one or more of pressure or force data and provide an alert as described herein. In some embodiments, the fluidic sensor comprises an atraumatic tip configured to deform upon engagement of tissue with resistance. The one or more sensors 530 may comprise any suitable sensor as described herein.

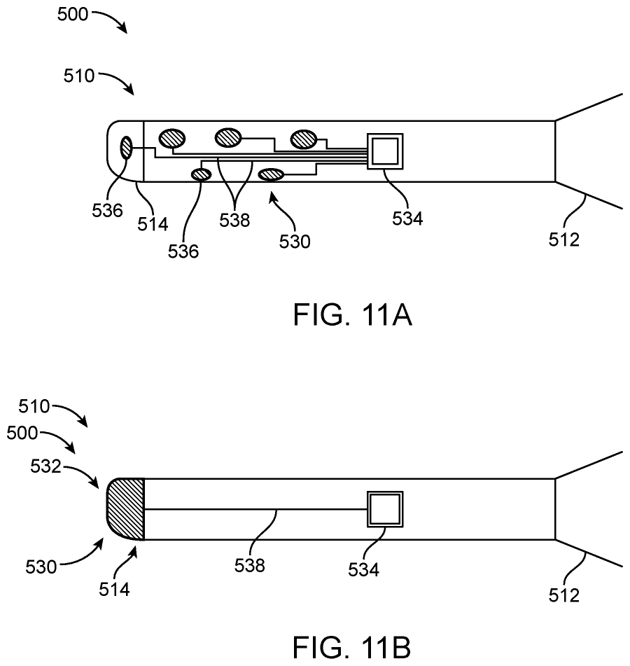

FIG. 12A shows a cover 560 comprising one or more sensors 530 configured for placement on a probe. In some embodiments, the cover 560 comprises a connector 562 to couple the cover the probe. In some embodiments, the cover 560 comprises a sheath 566 comprising a membrane material, such as a compliant or a non-compliant membrane material.

FIG. 12B shows a probe 510 configured to receive a cover as in FIG. 12A as indicated with arrow 1201. In some embodiments, the probe comprises a connector 564 to couple the cover to the probe.

FIG. 12C shows a cover 560 as in FIG. 12A placed over a probe 510.

Referring collectively to FIGS. 12A to 12C, the cover can be configured to measure pressure related to tissue resistance with insertion of the probe into tissue as described herein. In some embodiments, the cover 560 is coupled to probe 510 with engagement between connector 562 and connector 564. The connectors may comprise a locking engagement, such as snap on connectors, for example. Alternatively or in combination, cover 560 can be retained on probe 510 with friction or an adhesive, for example. The probe 510 with cover 560 placed thereon may comprise any suitable structure of the embodiments of probe 510 as described herein.

In some embodiments, cover 560 comprises a pressure sensor such as fluidic sensor 532 or other suitable sensor as described herein. The sensor can be coupled to one or more lines 538 extending proximally from the distal portion 514 toward the proximal portion 512. In some embodiments, the one or more lines 538 extends to a proximal connector or transducer 534 located on a proximal portion of cover 560.

In some embodiments, the proximal connector or transducer is configured to couple to the processor and provide one or more of alerts, alarms or mapping as described herein. In some embodiments, the cover is configured to provide resistive force feedback to the user as described herein.

While the cover 560 can be configured in many ways, in some embodiments the one or more sensors 530 can be configured to measure pressure with the cover placed on the probe. In some embodiments, the cover 560 comprises a disposable probe cover that can be removed after the probe has been inserted and removed from the patient with the cover placed thereon. In some embodiments, the cover 560 comprises a fluid-filled chamber with a compliant membrane as described herein. In some embodiments, one or more lines 538 comprise fluidic lines providing fluidic communication between the pressure sensor and a pressure transduce 534 proximal to the pressure sensor such as fluidic sensor 532.

The one or more lines 538 can be configured in many ways. In some embodiments, the one or more lines comprises a tube extending in a distal proximal direction along the cover 560.

In some embodiments, the measured force from the pressure is sensor is provided to the user, for example with a display.

In some embodiments, transducer 534 is located on a proximal portion 512 of the probe 510 and is connected to the fluidic pressure sensor with a connector on the tube. Alternatively, the pressure transducer may comprise a disposable pressure transducer could be built into the distal chamber and coupled to the proximal portion 512 of the probe with a connector such as an electrical connector. In some embodiments, electrical contacts are provided on the proximal portion 512 of probe 510 and operatively coupled to the processor for communication with the connector and processor such as a signal processor.

The cover 560 can be configured in many ways and can cover probe 510 as described herein and may comprise one or more components of probe 510 as described herein. In some embodiments, the cover 560 is configured to detect resistance of the probe 510 while being inserted into the patient. In some embodiments, the cover 560 comprises an elongate sheath 566 comprising a distal portion and a proximal portion configured to be placed on the corresponding distal portion 514 and corresponding proximal portion 512 of the probe 510. The one or more sensors 530 is supported with the sheath to detect a tissue resistance of the probe related to advancement of the probe into the patient.

In some embodiments, the cover comprises an interface such as connector or transducer 534 configured to couple the one or more sensors 530 to an output configured to provide an alert to a user in response to the tissue resistance.

In some embodiments the cover 560 comprises one or more lines 538 such as one or more channels, extending from the one or more sensors to the interface. The one or more lines may comprise one or more of a fluidic channel, an electrical conductor, or an optical fiber to couple the one or more sensors to the interface. In some embodiments, the interface comprises one or more of a transducer 534 or a connector configured to couple to a corresponding interface of a system to treat or image the patient.

In some embodiments, the one or more sensors 530 are configured to be inserted into the patient.

In some embodiments, the one or more sensors 530 are located on a superior side over the distal portion 514, which can be helpful with some tissue folds which tend to engage the superior side of the probe, such as a transrectal ultrasound probe.

In some embodiments, the one or more sensors 530 are located on the distal portion the sheath to cover the distal portion 514 of the probe to engage the tissue and detect the resistance. In some embodiments, the one or more sensors 530 of the cover 560 comprises a plurality of sensors. In some embodiments, the plurality of sensors of cover 530 is configured to detect tissue resistance at a plurality of locations on the distal portion of sheath covering the distal portion of the probe 510. In some embodiments, the plurality of sensors on the cover 530 is configured to map the tissue resistance to the probe at the plurality of locations as described herein.

In some embodiments, the one or more sensors 530 of the cover 560 comprise a switch to generate the output in response to the tissue resistance.

The one or more sensors 530 of the cover 560 can be configured in any suitable way and may comprise one or more of a force sensor, a pressure sensor, a fluidic sensor, a membrane, a membrane coupled to a fluid, a fluidic channel, a fluidic channel coupled to an electrical detector, a fluidic channel coupled to an electrical switch, a fluidic channel coupled to a pressure sensor, a fluidic sensor coupled to a proximal balloon, force sensor, a pressure sensor, a piezo electric sensor, a hall effect sensor, a capacitive sensor, an optical sensor, a strain gauge, a displacement transducer, a linear voltage displacement transducer (LVDT), an optical sensor, a light emitting diode, a diode laser, a photo diode, a photo transistor, or a quadrant photo detector.

In some embodiments, the cover 560 comprising one or more sensors 530 comprising an annular ring located on a distal portion of the sheath corresponding to the distal portion 514 of the probe. In some embodiments, the annular ring is located concentrically relative to an elongate axis of the probe.

While the one or more sensors 530 on the cover 560 can be configured in many ways, in some embodiments, the one or more sensors 530 comprises one or more pressure sensors such as fluidic sensors 532 located near the distal portion of the cover, the one or more sensors 532 comprising one or more membranes coupled to one or more proximally extending fluidic channel. In some embodiments, the one or more fluidic sensors comprises a plurality of fluidic sensors, the one or more membranes comprises a plurality of membranes, and the one or more proximally extending fluidic channels comprises a plurality of proximally extending fluidic channels. In some embodiments, the cover comprises a pressure sensor coupled to the one or more proximally extending fluidic channels to detect the resistance in response to a fluidic pressure of the one or more proximally extending fluidic channels. In some embodiments, the one or more fluidic sensors 532 comprises a tip of the sheath configured to cover distal portion 514 of the probe, the tip comprising a membrane containing a fluid located on the tip to soften the tip. Although reference is made to fluidic pressure sensors and fluidic channels, the sensors may comprise any suitable sensors as described herein such as pressure sensors and the plurality of channels may comprise any suitable channels such as electrical signal lines, optical fibers, etc.

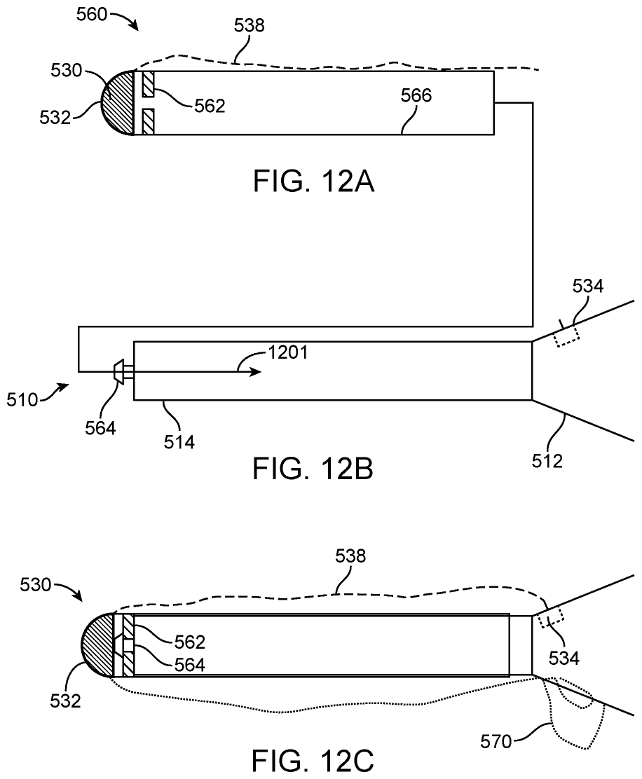

FIG. 13 shows a probe 510 in which the one or more sensors 530 is configured to provide mechanical tactile feedback in response to force associated with tissue resistance 590. The distal portion 514 of the probe comprises a soft material 543 such as an elastomer configured to deform and urge a diaphragm 595 from a first configuration 596 corresponding to no substantial tissue resistance to a second configuration 597 corresponding to substantial tissue resistance.

The diaphragm 595 can be configured in any suitable way and may comprise any suitable material. In some embodiments, the diaphragm 595 comprises a clicker.

In some embodiments, the diaphragm comprises a metal diaphragm spring configured to deliver an audible or tactile click that is perceived by the user when a force threshold is exceeded. In some embodiments, the tip of distal portion 514 is connected to the rest of the shaft via a diaphragm spring with soft elastomer 514 filling the gap. Alternatively, the one or more sensors may comprise an angular clicker, similar to a torque wrench mechanism, as will be understood by one or ordinary skill in the art.

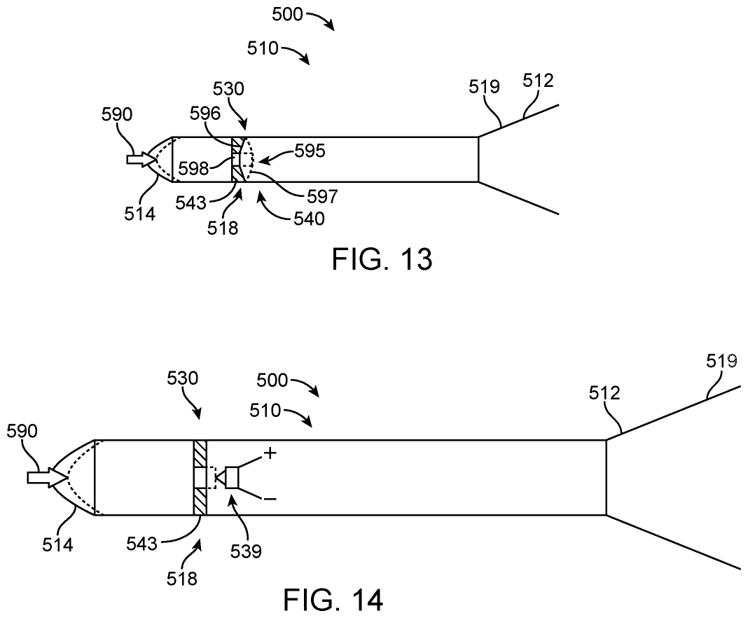

FIG. 14 shows a probe 510 in which the one or more sensors 530 comprises a switch 539 configured to transition between open and closed configurations in response to tissue resistance. The switch 539 may comprise components such as material 543 as described herein, in order to allow the switch to change states in response to force associated with tissue resistance as described herein.

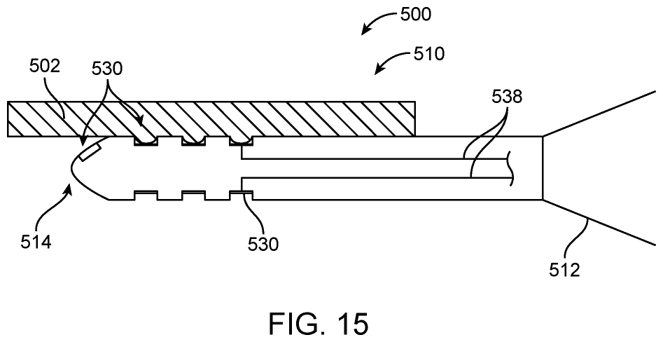

FIG. 15 shows a probe 510 comprising one or more sensors 530 configured to measure tissue impedance or proximity and detect changes in tissue impedance or proximity in response to forces related to mechanical tissue resistance while the probe is being advanced. In some embodiments, the one or more sensors 530 are connected to impedance circuitry or one or more transducers with one or more lines 538. In some embodiments, the one or more lines comprise electrically conductive lines. In some embodiments, the one or more sensors comprises one or more electrodes to measure tissue impedance at one or more locations. In some embodiments, the one or more electrodes comprise one or more recessed rings with a conductor at the bottom of a trough to measure one or more of impedance or proximity of the tissue in response to the tissue pressed against the probe. In some embodiments, proximity of tissue is related to capacitance associated with proximity of tissue to the electrodes. Alternatively or in combination, the impedance of the tissue can be measured by passing an alternating current through the tissue. Work in relation to the present disclosure suggests that tissue impedance can be related to pressure to the tissue, and changes in impedance used to detect pressure on the probe associated with tissue engaging the probe.

The presently disclosed cover can be configured in many ways and may comprise any suitable stiffness. In some embodiments, the sheath comprises a stiff sheath configured for placement in the patient prior to covering a probe. For example, the stiff sheath may comprise columnar strength sufficient to advance the sheath into a body lumen of the patient without support from a probe. In some embodiments, the stiff sheath is sized to receive the probe subsequent to insertion into the body lumen of the patient. Alternatively or in combination, wherein the sheath may be configured to be placed over a probe prior to insertion into a patient. In some embodiments, the sheath comprises a soft material configured to deform without support from a probe within the sheath.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A probe for insertion into a patient, the probe comprising: an elongate body comprising a distal portion and a proximal portion, the distal portion shaped for insertion into the patient, the proximal portion coupled to the distal portion to advance the distal portion with advancement of the proximal portion; one or more sensors supported with the elongate probe body and coupled to the distal portion of the probe to detect a tissue resistance of the distal portion related to advancement of the proximal portion; and an output operatively coupled to the one or more sensors to provide feedback to a user in response to the tissue resistance.

Clause 2. The probe of clause 1, wherein the one or more sensors are configured to be inserted into the patient.

Clause 3. The probe of clause 1, wherein the one or more sensors are located on a superior side of the distal portion when the probe has been inserted into the patient.

Clause 4. The probe of clause 1, wherein the one or more sensors are located on the distal portion of the probe to engage the tissue and detect the resistance.

Clause 5. The probe of clause 1, wherein the one or more sensors comprises a plurality of sensors.

Clause 6. The probe of clause 5, wherein the plurality of sensors is configured to detect tissue resistance at a plurality of locations on the distal portion of the probe.

Clause 7. The probe of clause 6, wherein the plurality of sensors is configured to map the tissue resistance to the probe at the plurality of locations.

Clause 8. The probe of clause 1, wherein the one or more sensors are configured to detect strain or compression of the elongate body between the first portion and the second portion in response to the tissue resistance.

Clause 9. The probe of clause 8, wherein the elongate body comprises a first portion and a second portion configured to move relative to the first portion and the one or more sensors is configured to detect movement of the first portion relative to the second portion.

Clause 10. The probe of clause 9, wherein the first portion is coupled to the second portion with a spring and wherein the second portion is configured to move relative to the first portion in response to a force from the tissue resistance greater than a force from the spring.

Clause 11. The probe of clause 10, wherein a spring is configured to push the first portion away from the second portion against a stop and wherein the first portion moves toward the second portion in the response to the tissue resistance force exceeding the force from the spring in order to decrease movement of the distal portion while the proximal portion advances.

Clause 12. The probe of clause 10, wherein the spring comprises one or more of a coil, a spring, a torsion spring, a leaf spring or a bendable extension.

Clause 13. The probe of clause 9, wherein the first portion and the second portion comprise telescopic portions and the one or more sensors is configured to detect relative movement between a first telescopic portion and a second telescopic portion.

Clause 14. The probe of clause 10, further comprising a switch configured to transition between an open configuration and a closed configuration and generate the output in response to the force from the resistance greater than the force from the spring.

Clause 15. The probe of clause 8, wherein the body comprises a shaft and wherein the distal portion is configured to move within the shaft in response to the tissue resistance.

Clause 16. The probe of clause 8, wherein the elongate body comprises a tapered waist between the distal portion and the proximal portion, and the one or more sensors are coupled to the tapered waist to detect strain of the tapered waist.

Clause 17. The probe of clause 8, wherein the one or more sensors is located between the distal portion and the proximal portion to detect the strain or compression of the elongate body in response to the resistance.

Clause 18. The probe of clause 8, wherein the one or more sensors comprises a displacement transducer to measure a displacement of the first portion relative to the second portion.

Clause 19. The probe of clause 8, wherein the elongate body extends along an elongate axis and the one or more sensors comprises a plurality of sensors located around the elongate axis to detect tissue resistance on a side of the probe away from the elongate axis.

Clause 20. The probe of clause 19, wherein the plurality of sensors located around the elongate axis are configured to detect a direction of deflection of the elongate body in response to the resistance.

Clause 21. The probe of clause 8, wherein the elongate body extends along an elongate axis and the one or more sensors comprises a plurality of sensors located at a first axial location corresponding to a first location along the elongate axis and a second axial location corresponding to a second location along the elongate axis, the second location different from the first location.

Clause 22. The probe of clause 1, wherein the probe comprises a switch to generate the output in response to the tissue resistance.

Clause 23. The probe of clause 1, wherein the alert comprises one or more of a sound, a sound that changes frequency in response to the tissue resistance, a sound that increases frequency in response to increased tissue resistance and decreases frequency in response to decreased tissue resistance, a message on a display, visible light indicator, a color on a display, a numeric value, a color bar, a vibration, a user perceptible vibration, or a break away structure to allow movement of the proximal end independently of the distal end.

Clause 24. The probe of clause 1, wherein the probe comprises a surgical probe to treat tissue.

Clause 25. The probe of clause 1, wherein the probe comprises an ultrasound probe.

Clause 26. The probe of clause 25, wherein the ultrasound probe comprises an array of ultrasound transducers to generate an image of the tissue.

Clause 27. The probe of clause 26, wherein the array of ultrasound transducers comprises a first array for transverse imaging and a second array for sagittal imaging.

Clause 28. The probe of clause 27, wherein the first array and the second array are located between the proximal portion and the distal portion and the one or more sensors is located between the proximal portion and the distal portion.

Clause 29. The probe of clause 28, wherein the one or more sensors are located between the first array and the second array.

Clause 30. The probe of clause 28, wherein the one or more sensors are not located between the first array and the second array.

Clause 31. The probe of clause 28, wherein the one or more sensors are located distal to the first array and the second array.

Clause 32. The probe of clause 28, wherein the one or more sensors are located proximal to the first array and the second array.

Clause 33. The probe of clause 1, wherein the one or more sensors comprises one or more of a force sensor, a pressure sensor, a fluidic sensor, a membrane, a membrane coupled to a fluid, a fluidic channel, a fluidic channel coupled to an electrical detector, a fluidic channel coupled to an electrical switch, a fluidic channel coupled to a pressure sensor, a fluidic sensor coupled to a proximal balloon, force sensor, a pressure sensor, a piezo electric sensor, a hall effect sensor, a capacitive sensor, an optical sensor, a strain gauge, a displacement transducer, a linear voltage displacement transducer (LVDT), an optical sensor, a light emitting diode, a diode laser, a photo diode, a photo transistor, a quadrant photo detector, a motion sensor, an accelerometer, an inertial measurement unit (IMU), Doppler ultrasound or tissue impedance sensor.

Clause 34. The probe of clause 33, wherein the one or more sensors comprises an annular ring located on the distal portion of the elongate body.

Clause 35. The probe of clause 34, wherein the annular ring is located concentrically relative to an elongate axis of the elongate body.

Clause 36. The probe of clause 33, wherein the one or more sensors comprises one or more sensors located near the distal portion of the probe, the one or more sensors comprising one or more membranes coupled to one or more proximally extending channels.

Clause 37. The probe of clause 36, wherein the one or more sensors comprises a plurality of sensors, the one or more membranes comprises a plurality of membranes, and the one or more proximally extending channels comprises a plurality of proximally extending channels.

Clause 38. The probe of clause 36, further comprising a pressure sensor coupled to the one or more proximally extending fluidic channels to detect the resistance in response to a fluidic pressure of the one or more proximally extending fluidic channels.

Clause 39. The probe of clause 36, wherein the one or more sensors comprises a tip of the distal portion, the tip comprising a membrane containing a fluid located on the tip to soften the tip.

Clause 40. The probe of clause 1, wherein the probe comprises a proximal handle for a user to advance and retract the probe.

Clause 41. A system comprising: the probe of clause 1; an arm coupled to the elongate body to support the elongate body; a proximal sensor operatively coupled to the arm and the elongate body to measure a displacement of the proximal portion of the probe; and a processor coupled to the one or more sensors and the proximal sensor to detect the tissue resistance of the distal end in response to the displacement of the proximal portion.

Clause 42. The system of clause 41, wherein the processor is configured to generate the output.

Clause 43. The system of clause 41, wherein the one or more sensors are configured to generate a signal in response to the tissue resistance and wherein the processor is configured to detect the tissue resistance in response to the signal and the displacement.

Clause 44. The system of clause 41, further comprising an actuator coupled to the probe and the arm, the actuator configured to advance and retract the probe, the sensor configured to measure the displacement in response to the actuator moving the probe.

Clause 45. The system of clause 44, wherein the actuator is configured to advance and retract the probe while the arm remains substantially fixed.

Clause 46. The system of clause 45, wherein the actuator comprises a user manipulable actuator configured for the user to advance and retract the probe with user manipulations.

Clause 47. The system of clause 46, wherein the actuator comprises a rotatable knob coupled to a rack and pinion.

Clause 48. The system of clause 41, wherein the arm comprises a robotic arm configured to advance and retract the probe.

Clause 49. The system of clause 41, wherein the probe comprises a proximal handle for a user to advance and retract the probe.

Clause 50. A cover to detect a tissue resistance during insertion into a patient, the cover comprising: an elongate sheath comprising a distal portion and a proximal portion; and one or more sensors supported with the sheath to detect the tissue resistance of related to advancement of the probe into the patient.

Clause 51. The cover of clause 50, further comprising an interface configured to couple the one or more sensors to an output configured to provide feedback to a user in response to the tissue resistance.

Clause 52. The cover of clause 51, further comprising one or more channels extending from the one or more sensors to the interface and optionally wherein the one or more channels comprise one or more of a fluidic channel, an electrical conductor, or an optical fiber to couple the one or more sensors to the interface.

Clause 53. The cover of clause 51, wherein the interface comprises one or more of a transducer or a connector configured to couple to a corresponding interface of a system to treat or image the patient.

Clause 54. The cover of clause 50, wherein the one or more sensors are configured to be inserted into the patient.

Clause 55. The cover of clause 50, wherein the one or more sensors are located on a superior side of the distal portion.

Clause 56. The cover of clause 50, wherein the one or more sensors are located on the distal portion of the probe to engage the tissue and detect the resistance.

Clause 57. The cover of clause 50, wherein the one or more sensors comprises a plurality of sensors.

Clause 58. The cover of clause 57, wherein the plurality of sensors is configured to detect tissue resistance at a plurality of locations on the distal portion of the sheath.

Clause 59. The cover of clause 58, wherein the plurality of sensors is configured to map the tissue resistance to insertion of the probe at the plurality of locations.

Clause 60. The cover of clause 50, wherein the sheath comprises a switch to generate the output in response to the tissue resistance.

Clause 61. The cover of clause 50, wherein the one or more sensors comprises one or more of a force sensor, a pressure sensor, a fluidic sensor, a membrane, a membrane coupled to a fluid, a fluidic channel, a fluidic channel coupled to an electrical detector, a fluidic channel coupled to an electrical switch, a fluidic channel coupled to a pressure sensor, a fluidic sensor coupled to a proximal balloon, force sensor, a pressure sensor, a piezo electric sensor, a hall effect sensor, a capacitive sensor, an optical sensor, a strain gauge, a displacement transducer, a linear voltage displacement transducer (LVDT), an optical sensor, a light emitting diode, a diode laser, a photo diode, a photo transistor, or a quadrant photo detector.

Clause 62. The cover of clause 61, wherein the one or more sensors comprises an annular ring located on the distal portion of the sheath.

Clause 63. The cover of clause 62, wherein the annular ring is located concentrically relative to an elongate axis of the probe.

Clause 64. The cover of clause 61, wherein the one or more sensors comprises one or more fluidic sensors located near the distal portion of the cover, the one or more fluidic sensors comprising one or more membranes coupled to one or more proximally extending fluidic channel.

Clause 65. The cover of clause 61, wherein the one or more fluidic sensors comprises a plurality of fluidic sensors, the one or more membranes comprises a plurality of membranes, and the one or more proximally extending fluidic channels comprises a plurality of proximally extending fluidic channels.

Clause 66. The cover of clause 61, further comprising a pressure sensor coupled to the one or more proximally extending fluidic channels to detect the resistance in response to a fluidic pressure of the one or more proximally extending fluidic channels.

Clause 67. The cover of clause 61, wherein the one or more fluidic sensors comprises a tip of the distal portion, the tip comprising a membrane containing a fluid located on the tip to soften the tip.

Clause 68. The cover of clause 50, wherein the sheath comprises a stiff sheath configured for placement in the patient prior to covering a probe.

Clause 69. The cover of clause 68 wherein the stiff sheath comprises columnar strength sufficient to advance the sheath into a body lumen of the patient without support from a probe and optionally wherein the stiff sheath is sized to receive the probe subsequent to insertion into the body lumen of the patient.

Clause 70. The cover of clause 50, wherein the sheath comprises a soft material configured to deform without support from a probe within the sheath.

Clause 71. The cover of clause 50, wherein the sheath is configured to be placed over a probe prior to insertion into a patient.

Clause 72. The probe, system or cover of any one of the preceding clauses wherein the output is operatively coupled to a processor and optionally wherein the processor comprises a signal processor.

Clause 73. A method of inserting a probe into tissue, the method comprising using the probe, system, or cover of any one of the preceding clauses.

Clause 74. A method of inserting a probe into a patient, the method comprising: inserting the probe into the patient; viewing sensor data from one or more sensors on the probe; and adjusting placement of the probe in response to the sensor data.

Clause 75. A method of coupling an ultrasound probe into a patient, the method comprising: viewing sensor data from one or more sensors coupled to the ultrasound probe; placing the probe in the patient in response to the sensor data.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A probe for insertion into a patient, the probe comprising:
an elongate body comprising a distal portion and a proximal portion, the distal portion shaped for insertion into the patient, the proximal portion coupled to the distal portion to advance the distal portion with advancement of the proximal portion;
one or more sensors supported with the elongate probe body and coupled to the distal portion of the probe to detect a tissue resistance of the distal portion related to advancement of the proximal portion; and
an output operatively coupled to the one or more sensors to provide feedback to a user in response to the tissue resistance.

2. The probe of claim 1, wherein the one or more sensors are configured to be inserted into the patient.

3. The probe of claim 1, wherein the one or more sensors are located on a superior side of the distal portion when the probe has been inserted into the patient.

4. The probe of claim 1, wherein the one or more sensors are located on the distal portion of the probe to engage the tissue and detect the resistance.

5. The probe of claim 1, wherein the one or more sensors comprises a plurality of sensors.

6. The probe of claim 5, wherein the plurality of sensors is configured to detect tissue resistance at a plurality of locations on the distal portion of the probe.

7. The probe of claim 6, wherein the plurality of sensors is configured to map the tissue resistance to the probe at the plurality of locations.

8. The probe of claim 1, wherein the one or more sensors are configured to detect strain or compression of the elongate body between the first portion and the second portion in response to the tissue resistance.

9. The probe of claim 8, wherein the elongate body comprises a first portion and a second portion configured to move relative to the first portion and the one or more sensors is configured to detect movement of the first portion relative to the second portion.

10. The probe of claim 9, wherein the first portion is coupled to the second portion with a spring and wherein the second portion is configured to move relative to the first portion in response to a force from the tissue resistance greater than a force from the spring.

11. The probe of claim 10, wherein a spring is configured to push the first portion away from the second portion against a stop and wherein the first portion moves toward the second portion in the response to the tissue resistance force exceeding the force from the spring in order to decrease movement of the distal portion while the proximal portion advances.

12. The probe of claim 10, wherein the spring comprises one or more of a coil, a spring, a torsion spring, a leaf spring or a bendable extension.

13. The probe of claim 9, wherein the first portion and the second portion comprise telescopic portions and the one or more sensors is configured to detect relative movement between a first telescopic portion and a second telescopic portion.

14. The probe of claim 10, further comprising a switch configured to transition between an open configuration and a closed configuration and generate the output in response to the force from the resistance greater than the force from the spring.

15. The probe of claim 8, wherein the body comprises a shaft and wherein the distal portion is configured to move within the shaft in response to the tissue resistance.

16. The probe of claim 8, wherein the elongate body comprises a tapered waist between the distal portion and the proximal portion, and the one or more sensors are coupled to the tapered waist to detect strain of the tapered waist.

17. The probe of claim 8, wherein the one or more sensors is located between the distal portion and the proximal portion to detect the strain or compression of the elongate body in response to the resistance.

18. The probe of claim 8, wherein the one or more sensors comprises a displacement transducer to measure a displacement of the first portion relative to the second portion.

19. The probe of claim 8, wherein the elongate body extends along an elongate axis and the one or more sensors comprises a plurality of sensors located around the elongate axis to detect tissue resistance on a side of the probe away from the elongate axis.

20. The probe of claim 19, wherein the plurality of sensors located around the elongate axis are configured to detect a direction of deflection of the elongate body in response to the resistance.
